(19) United States
(12) Patent Application Publication
(10) Pub. No.: US 2006/0003381 A1
(43) Pub. Date: Jan. 5, 2006

(54) Methods for assembling protein microarrays

(76) Inventors: James Gilmore, Carlsbad, CA (US); Steven Daniel, Carlsbad, CA (US); Mike Hogan, Tucson, AZ (US); Sunny Tam, Westborough, MA (US); Rick Wiese, Roscoe, IL (US)

Correspondence Address: SENNIGER POWERS, ONE METROPOLITAN SQUARE, 16TH FLOOR, ST LOUIS, MO, 63102, US

(21) Appl. No.: 10/476,739

(22) PCT Filed: May 3, 2002
(86) PCT No.: PCT/US02/13868

§ 371 Date: Jun. 9, 2005

Related U.S. Application Data
(60) Provisional application No. 60/288,688, filed on May 3, 2001.

(51) Int. Cl. G01N 33/53; G01N 33/00; C12M 1/34; A61L 2/00

(52) U.S. Cl. 435/7.1; 435/287.2; 436/86; 427/2.11

(57) ABSTRACT

The invention provides methods for assembling protein arrays. In one aspect, the methods comprise use of charged or polar array surfaces in the construction of the array. The methods also comprise use of viscous solutions for the deposition of the polypeptides on the array surface in the construction of the array.

This application claims the benefit of U.S. Provisional Application No. 60/288,688, filed May 3, 2001.

### TECHNICAL FIELD

This invention relates generally to cell biology, proteomics and polypeptide array, or “biochip,” technology. In particular, the invention is directed to methods for assembling protein arrays. In one aspect, the methods comprise use of charged or polar array surfaces in the construction of arrays. The methods also comprise use of viscous solutions for the deposition of the polypeptides on the array surface in the construction of arrays.

### BACKGROUND

One class of protein microarray uses immobilized “capture antibody.” The polypeptides are bound to a solid substrate, such as glass with a treated surface, such as aminosilane. Polypeptides are commonly bound to the substrate through a biotin-streptavidin conjugation. This requires modification of the substrate, a process that can be difficult, non-specific and expensive. The arrays are then incubated with a solution containing antigen that will bind to the capture antibodies in a manner dependent upon time, buffer components, and recognition specificity. The antigens may then be visualized directly if they have been previously labeled, or may be allowed to bind to a secondary labeled reagent, frequently another antibody. The means of visualizing the amount of antigen bound to the capture antibody is dependent upon the labeling method utilized, but is often by a CCD imager or laser scanner using filter sets that are appropriate to excite and detect the emissions of the label. The imager converts the amount of detected photons into an electronic signal (often an 8-bit or 16-bit scale) which can then be analyzed using software packages.

A major challenge in fabricating protein arrays is the ability to bind a polypeptide (e.g., the antibody or antigen) to the surface of the array, while still retaining biological activity.

### SUMMARY

The invention provides a method for assembling protein arrays comprising the following steps: (a) providing an array comprising a surface comprising a net positive or a net negative charge; (b) providing a solution comprising a polypeptide comprising a net positive (cationic) charge density or a net positive charge polarity or a net negative (anionic) charge density or a net negative charge polarity at its amino terminal end or its carboxy terminal end; and, (c) adding the solution to the array under conditions allowing the positive or negative end of the protein aligns with the negative or positive charge of the array surface.

In one aspect of the method, the array surface comprises a net negative (anionic) charge density or a net negative charge polarity and the polypeptide comprises a net positive (cationic) charge density or a net positive charge polarity. In one aspect, hydroxyl groups on the array surface provide a net negative (anionic) charge density or a net negative charge polarity. In one aspect sulfhydryl groups on the array surface provide a net negative (anionic) charge density or a net negative charge polarity.

In one aspect of the method, the array surface comprises a net positive (cationic) charge density or a net positive charge polarity and the polypeptide comprises a negative (anionic) charge density or a net negative charge polarity. In one aspect, the array surface comprises a net positive (cationic) charge density or a net positive charge polarity due to a plurality of charged amino groups on the array surface.

The invention provides a method for assembling protein arrays comprising the following steps: (a) providing an array; (b) providing a solution comprising a polypeptide, wherein the solution comprises a viscosity sufficient to provide surface tension such that the polypeptide-containing solution does not spread over an array surface area larger than about 200 nm in diameter; and, (c) adding the solution to the array.

The invention provides a method for assembling protein arrays comprising the following steps: (a) providing an array; (b) providing a solution comprising a polypeptide, wherein the solution comprises a viscosity of between about 1 mN*s/m² to about 30 mN*s/m²; and, (c) adding the solution to the array.

In one aspect, the solution comprises an organic polymer, such as a water soluble polymer (see discussion below for a complete discussion of polymers that can be used in the solutions of the methods of the invention). In alternative aspects, the solution comprises glycerol or polyethylene glycol. In alternative aspects, the solution comprises sodium azide or sodium iodide. The sodium azide concentration can be between about 0.2% and about 0.5%.

The details of one or more embodiments of the invention are set forth in the accompanying drawings and the description below. Other features, objects, and advantages of the invention will be apparent from the description and drawings, and from the claims.

All publications, patents, patent applications, GenBank sequences and ATCC deposits cited herein are hereby expressly incorporated by reference for all purposes.

### DESCRIPTION OF DRAWINGS

FIG. 1 schematically sets forth a map of an array used in the exemplary methods described in Example 1.

FIG. 2 is an illustration representing an array image demonstrating specificity and standard curves, as described in Example 1.

FIG. 3A is a linear regression graph and equation derived as set forth in Example 1, below. FIG. 3B is an antigen concentration graph and standard curves from data derived from application of sample to an array, as described in detail in Example 1, below

Like reference symbols in the various drawings indicate like elements.

### DETAILED DESCRIPTION

The invention provides novel methods for fabricating polypeptide arrays.

In one aspect of the invention, a charged or electrostatically polar array surface is used such that the surface can oriented onto the array charged or polar polypeptides such that the polypeptides are aligned upon binding to the array. This also improves binding kinetics. Thus, in one aspect of the invention of the methods of the invention, a polypeptide array is fabricated using a surface that electrostatically interacts with substrate (see, e.g., co-pending PCT/US00/23438, and U.S. patent application Ser. No. 09/636,268; describing electrostatically “tunable” surfaces). In one aspect, the substrate surface may be modified with a positive or a negative charge. For example, the substrate itself, or, polypeptides or other molecules immobilized onto the array surface, can comprise a net negative charge, e.g., by comprising hydroxy moieties at selected amino-acid positions. The array substrate modification is selectively chosen such that the substrate binds to polypeptides to be immobilized onto the array in a specific orientation (i.e., amino terminal versus carboxy terminal). Conversely, the polypeptides to be immobilized onto the array are designed to have a positive or negative charge, depending on the charge of the array substrate surface and the desired orientation of the polypeptide onto the surface.

In one aspect of the invention the polypeptides are added to the array in a “hydrating buffer” that does not interfere with printing or complementation. It is a significant improvement in the development of protein microarrays, as this allows the substrate to remain in a hydrated state during storage. In one aspect, the “hydrating buffer” comprises polymers that give the solution a viscosity sufficient to provide surface tension such that the polypeptides do not spread over an area larger than about 200 nm in diameter. In another aspect of the invention, the “hydrating buffer” comprises polymers that give the solution a viscosity of between about 1 to 30 mN*s/m².

One skilled in the art can use routine screening to select alternative reagents and polymers to prepare solutions with a viscosity of between about 1 to 30 mN*s/m², or, a solution a viscosity sufficient to provide surface tension such that the polypeptides do not spread over an area larger than about 200 nm in diameter that can be used in the methods of the invention. See, e.g., Nishida (2000) Eur. J. Pharm. Biopharm. 50:397-402; Sato (2000) Protein Sci. 9:1601-1603; Freerksen (1990) Anal. Biochem. 189:163-168; Danish (1983) J. Lab. Clin. Med. 101:515-526; Kawahara (1966) J. Biol. Chem. 241:3228-3232; and U.S. Pat. Nos. 6,215,033; 6,214,956; 6,196,058; 6,023,961. 5,644,395; 5,203,203; 4,862,735; 4,852,388; 4,704,898; 4,667,519; 4,499,753; 4,388,823.

These buffers can comprise any organic polymer, e.g., a glycogen or a polyethylene-glycol. Polyethylene glycol (PEG) with different MWs, e.g. PEG 400, PEG 800, PEG 1000, PEG 1,500, PEG 2,000, PEG 4,000, PEG 8,000 and PEG 20,000, and the like, and mixtures thereof can be used. Any water soluble polymers can be used, such as polyvinylpyrrolidone (e.g. PVP K30, K90 from BASF), hydroxyethylcellulose, hydroxypropylmethylcellulose, polyethylene oxide polymer (e.g., Polyox WSR-303™), hydroxypropylcellulose, carboxyvinyl polymers (Carbopol 940™), and the like; sugar alcohols such as D-sorbitol, mannitol, and the like; sugars such as sucrose, glucose, D-fructose, and the like; polyethylene-polyoxypropylene glycol, polyethylene-sorbitan fatty acid ester, salt, and the like.

Other compositions can also be included in these buffers, e.g., sodium iodide, sodium azide.

An additional improvement is the development of a surface chemistry that utilizes native substrate rather than biotin-streptavidin conjugation to bind the substrate to the surface.

Any type of array surface can be used in the methods of the invention, e.g., the method can utilize a silanized surface. The array substrate surface can comprise any polypeptide or peptide, e.g., a “capture antibody,” an antigen, an antigen that is bound to an antibody, and the like. These polypeptides can bind covalently or non-covalently to the array substrate surface (e.g., silanized surface).

Definitions

Unless defined otherwise, all technical and scientific terms used herein have the meaning commonly understood by a person skilled in the art to which this invention belongs. As used herein, the following terms have the meanings ascribed to them unless specified otherwise.

The terms “array” or “microarray” or “protein array” or “proteome array” or “biochip” as used herein are used interchangeably herein, and include all known variations of these devices, as discussed in detail, below.

By “biosite” is meant the biological molecules or capture probes (e.g., polypeptides) that are deposited on the surface of a reaction substrate, or base material, of an array. Under appropriate conditions, an association, e.g., a specific binding, or hybridization, can occur between the probe and a target molecule. The components of the biological molecule form the biosite since there is the potential of an interaction or a reaction occurring between each component strand of the biological molecule and the target molecule. The maximum number of biosites per array will depend on the size of the array, or reaction vessel within an array, may vary, depending on the probe deposition technology (e.g., printing), the nature of the probe, the means used to assess binding and/or to determine the volume or shape of a biosite (for quality control). For example, the size of a biosite on an array may depend on the practical optical resolution of the accompanying detector/imager. For example, an array of 16 (4×4 array) biosites may be deposited on the hybridization substrate or base material that eventually forms the bottom of the entire reaction vessel. In this example, each biosite may comprise a circle of approximately about 25 to 200 microns (μm) in diameter. Thus, for a 16 biosite array, each of the 16×200 μm diameter area contains a uniform field of probes attached to the hybridization substrate (base material) in a concentration which is highly dependent on the probe size and the well size. Each 25 to 200 μm diameter area can contain millions of probe molecules. Also, each of the 16 different biosites (probe sites) can contain one type of probe. Thus, 16 different probe types can be assayed in an array containing 16 biosites (4×4 array) per reaction chamber. As another example, four separate 10×10 arrays (400 biosites) can be generated to fit into one well of a 96 well microtiter plate with sufficient spacing between each of the 400 biosites. For this 10×10 format, 400 hybridization experiments are possible within a single reaction chamber corresponding to 38,400 (96×400) assays/hybridization that can be performed nearly simultaneously.

By “substrate” is meant the substrate that the polypeptides are deposited on, e.g., in the form of biosites. “Substrates” can be selected from a variety of materials, without limitation, e.g., polyvinyl, polystyrene, polypropylene, polyester, vinyl, other plastics, glass, SiO₂, other silanes, nylon membrane, gold or platinum, see further examples described, below. The solid surfaces can be derivatized, e.g., thiol-derivatized biopolymers and organic thiols can be bound to a metal solid substrate; see, e.g., U.S. Pat. No. 5,942,397 (see below for more examples). See, e.g., PCT/US00/23438, and U.S. patent application Ser. No. 09/636,268; describing electrostatically “tunable” surfaces that can be used in the methods of the invention.

The term “immobilized” means that the probe can be attached to a surface (e.g., the substrate) in any manner or any method; including, e.g., reversible or non-reversible binding, covalent or non-covalent attachment, and the like.

The term “solution” means a liquid or semi-liquid that is comprised of varying buffers and/or sample(s) and is applied to the array.

The term “antibody” refers to a peptide or polypeptide substantially encoded by an immunoglobulin gene or immunoglobulin genes, or fragments or equivalents thereof, capable of specifically binding an epitope, see, e.g. Fundamental Immunology, Third Edition, W. E. Paul, ed., Raven Press, N.Y. (1993); Wilson (1994) J. Immunol. Methods 175:267-73; Yarmush (1992) J. Biochem. Biophys. Methods 25:85-97. One of skill will appreciate that antibody fragments may be isolated or synthesized de novo either chemically or by utilizing recombinant DNA methodology. The term antibody also includes “chimeric” antibodies either produced by the modification of whole antibodies or those synthesized de novo using recombinant DNA methodologies. Typically, such chimeric antibodies are “humanized antibodies,” i.e., where the epitope binding site is generated from an immunized mammal, such as a mouse, and the structural framework is human. Immunoglobulins can also be generated using phage display libraries, and variations thereof. Antibodies or other molecules that bind to post-translationally modified polypeptides are well known in the art, see, e.g., U.S. Pat. Nos. 6,008,024; 5,763,198; 5,599,681; 5,580,742. Methods of producing polyclonal and monoclonal antibodies are known to those of skill in the art and described in the scientific and patent literature, see, e.g., Coligan, CURRENT PROTOCOLS IN IMMUNOLOGY, Wiley/Greene, NY (1991); Stites (eds.) BASIC AND CLINICAL IMMUNOLOGY (7th ed.) Lange Medical Publications, Los Altos, Calif.; Goding, MONOCLONAL ANTIBODIES: PRINCIPLES AND PRACTICE (2d ed.) Academic Press, New York, N.Y. (1986); Harlow (1988) ANTIBODIES, A LABORATORY MANUAL, Cold Spring Harbor Publications, New York.

Nucleic Acid and Polypeptide Probes

This invention provides an array comprising immobilized polypeptides (other molecules, such as nucleic acids or oligonucleotides and polysaccharides, lipids or small molecules, can also be immobilized). For example, a polypeptide can be immobilized to an array substrate surface by conjugation to an oligonucleotide, which in turn specifically hybridizes to a nucleic acid immobilized on the array surface (see, e.g., U.S. Pat. No. 6,083,763). These probes can be made and expressed in vitro or in vivo, any means of making and expressing polypeptides or nucleic acids used in the devices or practiced with the methods of the invention can be used. The invention can be practiced in conjunction with any method or protocol known in the art, which are well described in the scientific and patent literature.

Techniques for the manipulation of nucleic acids and generating recombinant polypeptide, such as, e.g., generating mutations in sequences, subcloning, labeling probes, sequencing, hybridization and the like are well described in the scientific and patent literature, see, e.g., Sambrook, ed., MOLECULAR CLONING: A LABORATORY MANUAL (2ND ED.), Vols. 1-3, Cold Spring Harbor Laboratory, (1989); CURRENT PROTOCOLS IN MOLECULAR BIOLOGY, Ausubel, ed. John Wiley & Sons, Inc., New York (1997); LABORATORY TECHNIQUES IN BIOCHEMISTRY AND MOLECULAR BIOLOGY: HYBRIDIZAON WITH NUCLEIC ACID PROBES, Part I. Theory and Nucleic Acid Preparation, Tijssen, ed. Elsevier, N.Y. (1993).

As used herein, “polypeptides” and “proteins” can include, e.g., amino acids, peptides, oligopeptide, polypeptides, peptidomimetics, other short polymers or organic molecules. When amino acids are used, alternative embodiment can use methyl esters because of commercial availability and the fact that they are not altered by the formation reactions (binding of the association surface to the support surface). “Peptidomimetics” include synthetic chemical compounds that have substantially the same structural and/or functional characteristics of the corresponding composition, e.g., the peptides, oligopeptides (e.g., oligo-histidine, oligo-aspartate, oligo-glutamate, poly-(his)₂(gly)₁, and poly-(his)₂(asp)₁), polypeptides, imidazole derivatives or equivalents used in the association surface of the invention. The mimetic can be either entirely composed of synthetic, non-natural analogues of amino acids, or, is a chimeric molecule of partly natural peptide amino acids and partly non-natural analogs of amino acids. The mimetic can also incorporate any amount of natural amino acid conservative substitutions as long as such substitutions also do not substantially alter the mimetic's structure and/or activity. Individual peptidomimetic residues can be joined by peptide bonds, other chemical bonds or coupling means, such as, e.g., glutaraldehyde, N-hydroxysuccinimide esters, bifunctional maleimides, N,N′-dicyclohexylcarbodiimide (DCC) or N,N′-diisopropyl-carbodiimide (DIC). Linking groups that can be an alternative to the traditional amide bond (“peptide bond”) linkages include, e.g., ketomethylene (e.g., —C(═O)—CH₂— for —C(═O)—NH—), aminomethylene (CH₂—NH), ethylene, olefin (CH═CH), ether (CH₂—O), thioether (CH₂—S), tetrazole (CN4—), thiazole, retroamide, thioamide, or ester (see, e.g., Spatola (1983) in Chemistry and Biochemistry of Amino Acids, Peptides and Proteins, Vol. 7, p 267-357, Marcell Dekker, NY).

Arrays, or “BioChips”

The invention provides methods for assembling protein arrays. Proteins can be immobilized onto a solid surface for binding directly or indirectly. The biosites may be arranged on the solid surface at different sizes and different densities. The methods of the invention can incorporate in whole or in part designs of arrays, and associated components and methods, as described, e.g., in U.S. Pat. Nos. 6,197,503; 6,174,684; 6,156,501; 6,093,370; 6,087,112; 6,087,103; 6,087,102; 6,083,697; 6,080,585; 6,054,270; 6,048,695; 6,045,996; 6,022,963; 6,013,440; 5,959,098; 5,856,174; 5,843,655; 5,837,832; 5,770,456; 5,723,320; 5,700,637; 5,695,940; 5,556,752; 5,143,854; see also, e.g., WO 99/51773; WO 99/09217; WO 97/46313; WO 96/17958; WO 89/10977; see also, e.g., Johnston (1998) Curr. Biol. 8:R171-R174; Schummer (1997) Biotechniques 23:1087-1092; Kem (1997) Biotechniques 23:120-124; Solinas-Toldo (1997) Genes, Chromosomes & Cancer 20:399-407; Bowtell (1999) Nature Genetics Supp. 21:25-32; Epstein (2000) Current Opinion in Biotech. 11:36-41; Mendoza (1999) “High-throughput microarray-based enzyme-linked immunosorbent assay (ELISA),” Biotechniques 27: 778-788; Lueking (1999) Protein microarrays for gene expression and antibody screening,” Anal. Biochem. 270:103-111; Davies (1999) “Profiling of amyloid beta peptide variants using SELDI protein chip arrays,” Biotechniques 27:1258-1261.

Probe Deposition Onto Substrate

The invention provides an array by immobilizing onto a substrate a plurality of biosites comprising polypeptides. Polypeptides can be “deposited” or immobilized” onto the substrate using any method or combination of methods known in the art e.g., piezo-electric, such as ink-jet, processes and systems, robotic deposition, photolithographic in-situ synthesis, use of microsyringes, or a continuous flow bundled microcapillary process (see, e.g., U.S. Pat. No. 6,083,763). Array fabrication methods that can be incorporated, in whole or in part, in the making or using of the invention include, e.g., those described in U.S. Pat. Nos. 6,197,503; 6,177,238; 6,164,850; 6,150,147; 6,083,763; 6,048,695; 6,010,616; 5,599,695; 5,919,523; 5,861,242; 5,770,722; 5,750,669; 5,143,854.

Substrate Surfaces

The arrays used in the methods of the invention can comprise substrate surfaces of a rigid, semi-rigid or flexible material. The substrate surface can be flat or planar, be shaped as wells, raised regions, etched trenches, pores, beads, filaments, or the like. Substrates can be of any material upon which a “capture probe” can be directly or indirectly immobilized. For example, suitable materials can include paper, glass (see, e.g., U.S. Pat. No. 5,843,767), ceramics, quartz or other crystalline substrates (e.g. gallium arsenide), metals, metalloids, polacryloylmorpholide, various plastics and plastic copolymers, Nylon™, Teflon™, polyethylene, polypropylene, poly(4-methylbutene), polystyrene, polystyrene/latex, polymethacrylate, poly(ethylene terephthalate), rayon, nylon, poly(vinyl butyrate), polyvinylidene difluoride (PVDF) (see, e.g., U.S. Pat. No. 6,024,872), silicones (see, e.g., U.S. Pat. No. 6,096,817), polyformaldehyde (see, e.g., U.S. Pat. Nos. 4,355,153; 4,652,613), cellulose (see, e.g., U.S. Pat. No. 5,068,269), cellulose acetate (see, e.g., U.S. Pat. No. 6,048,457), nitrocellulose, various membranes and gels (e.g., silica aerogels, see, e.g., U.S. Pat. No. 5,795,557), paramagnetic or superparamagnetic microparticles (see, e.g., U.S. Pat. No. 5,939,261) and the like. The substrate can be derivatized for application of other compounds upon which the probes are immobilized. Reactive functional groups can be, e.g., hydroxyl, carboxyl, amino groups or the like. Silane (e.g., mono- and dihydroxyalkylsilanes, aminoalkyltrialkoxysilanes, 3-aminopropyl-triethoxysilane, 3-aminopropyltrimethoxysilane) can provide a hydroxyl functional group for reaction with an amine functional group.

Detection Probes and Devices

The polypeptides on the array, or the performance of a completed array, can be detected using any variety of methods and devices, including, e.g., use of radioactive, calorimetric, bioluminescent, fluorescent or chemiluminescent or another photon detectable moieties. “Detectable moieties,” such as fluorescent, bioluminescent or chemiluminescent, or radiation, can be detected and quantified, e.g., using assays, devices or imaging systems well known in the art, as described in, e.g., U.S. Pat. Nos. 6,225,670; 6,211,524; 6,198,835; 6,197,928; 6,197,499; 6,194,731; 6,194,223; 6,191,852; 6,191,425; 6,132,983; 6,087,476; 6,060,261; 5,866,348; 5,094,939; 5,744,320; 5,631,734; 5,192,980; 5,091,652.

For example, one imaging system can be a proximal charge-coupled device (CCD) detection/imaging; due to its inherent versatility, it can also accommodate chemiluminescence, fluorescent and radioisotope target molecule detection, high throughput, and high sensitivity. This detection/imaging apparatus can include a lensless imaging array comprising a plurality of solid state imaging devices, such as an array of CCDs, photoconductor-on-MOS arrays, photoconductor-on-CMOS arrays, charge injection devices (CIDs), photoconductor on thin-film transistor arrays, amorphous silicon sensors, photodiode arrays, or the like. The devices and methods of the invention incorporate in whole or in part designs of detection devices as described, e.g., in U.S. Pat. Nos. 6,197,503; 6,197,498; 6,150,147; 6,083,763; 6,066,448; 6,045,996; 6,025,601; 5,599,695; 5,981,956; 5,698,089; 5,578,832; 5,632,957.

### EXAMPLES

The following examples are offered to illustrate, but not to limit the claimed invention.

### Example 1

### Methods for Assembling a Polypeptide Array

Exemplary methods for assembling an array are provided. The array used for these experiments is configured as an 8×8 array of printed antibody, one array per well in a standard 8×12 microtiter format; the specific array design is illustrated in FIG. 1. The 64-element array contained a 5 element dilution series in duplicate for both forms of PSA and a 4 element dilution series printed in duplicate for IL-6. The rabbit IgG markers printed in positions A1-A8 and H-7 and 8 are useful for the orientation and identification of probes within the array.

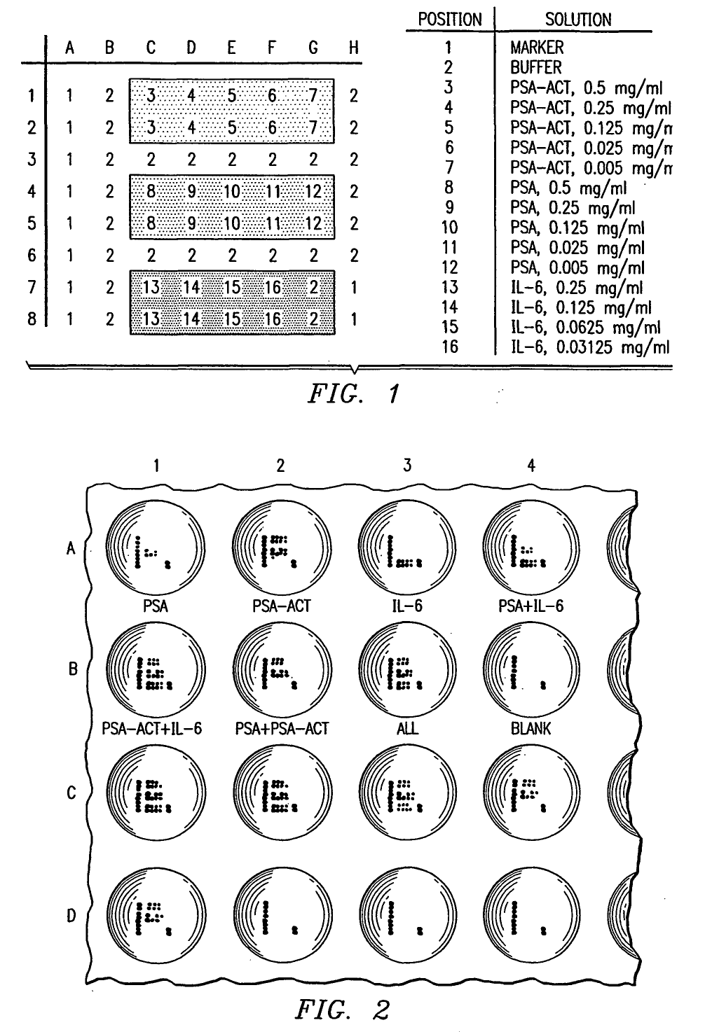

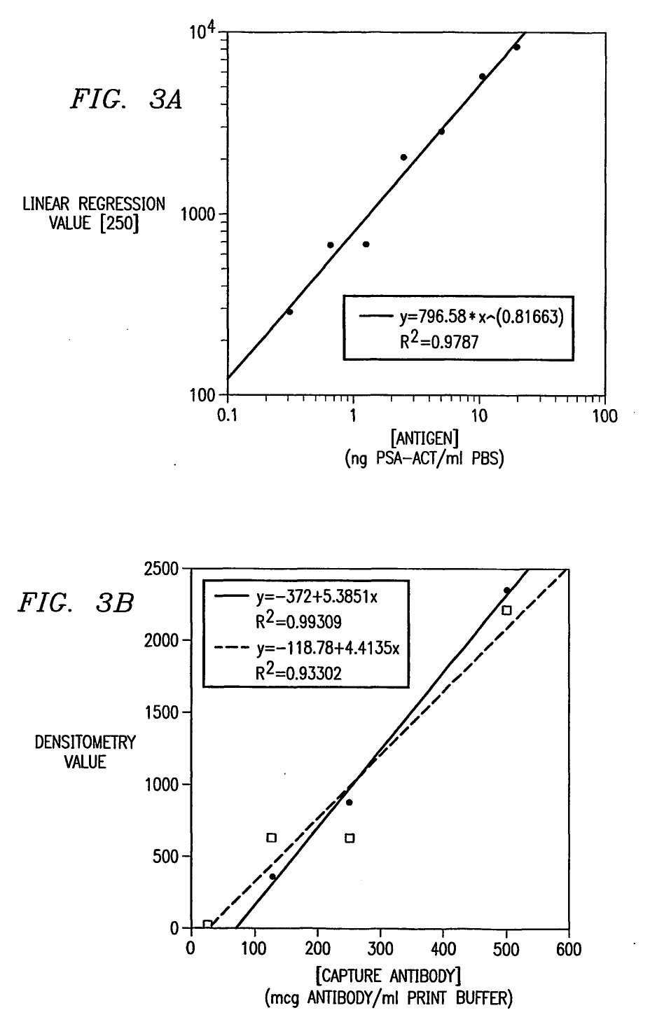

FIG. 2 is an image of 16 wells, which demonstrates the selectivity of the antibodies for the appropriate antigen (A1-B3), and contains the 7-point standard curve assayed in tandem for the 3 proteins of interest (C1-D3). Wells B4 and D4 are both negative controls (no recombinant protein added). As expected, in well A1 (PSA only) signal is detected only at the total PSA capture probes, in A2 (PSA-ACT) signal is detectable at both the total PSA and specific ACT bound PSA capture probes, and in A3 (IL-6) detectable signal emanates solely from the IL-6 probes. Additionally, for each of the combinations of these substrates only those probes specific for the added antigen yield a detectable signal (A4-B3). Densitometry values obtained from the standard curve wells (C1-D3) were used to construct a graph of densitometry value versus capture (printed) antibody concentration for each of the 3 antigens examined in each well for each antigen concentration, an example of one of these graphs is shown in FIG. 3B. The linear regression equations derived from each of these graphs were then used to derive the points for the linear regression value versus antigen concentration graph (standard curves) shown in FIG. 3A. For the total PSA curve the highest concentration is omitted so upper limits will match on both PSA forms (PSA total concentration is sum of PSA and PSA-ACT so the titration curve for detectable antigen actually covers the range 40 ng/ml to 0.625 ng/ml for the total PSA antibody). The correlation coefficients derived from the regression lines are comparable, if not superior, to those attained utilizing standard ELISA.

This multiplex microELISA system allows for savings of materials and time in the construction of standard curves and the analysis of samples compared to traditional ELISA due to the fact that the standard curves can be run simultaneously (all analytes in a single well) instead of single or replicate wells for each concentration of each antigen or sample. In addition, to time and sample savings (only 25 μl of sample is needed), capture antibody usage is decreased in this system as well. As an example, 40 μl of the IL-6 capture antibody would be necessary to prepare one 96 well microtiter plate for standard ELISA according to the manufacturers recommended dilutions. Performing protein quantification by this microELISA system, utilizing array construction by capillary printer6 it is possible to print more than a hundred 96 well arrays with this same 40 μl of capture antibody.

The information available from each well is significantly greater in this microarray configuration as compared to a standard ELISA as well. In standard ELISA, the values used to determine analyte concentration are 3 sample absorbance values (if the test is performed in triplicate), here the number of data points used to determine these concentrations are often twice that number and no less then equal to it at the lower analyte concentrations, utilizing a single well and multiple antibody dilutions printed in duplicate. The use of a capture antibody dilution series allows for a greater working range in our ELISA format as well. As the antigen concentration increases lower capture antibody concentration probes are detectable, and as the higher detection probe concentrations become saturated the lower probe concentrations can be used for quantification. This factor virtually eliminates the necessity of having to dilute samples and repeat an assay; this is especially valuable when working with limited sample amounts. As an example of this, the PSA (total) array is capable of detecting PSA at concentrations up to 100 ng/ml (data not shown). Additionally, this array design is not constrained by the need to analyze proteins present within the sample at approximately equal concentrations. In the experiments reported here, there is approximately a 500-fold difference in protein concentrations from highest (PSA, 20 ng/ml) to lowest (IL-6, 0.0046875 ng/ml), other work we have completed has demonstrated a range of approximately 400,000-fold (2 mg/ml to 4 pg/ml).

This system of microarray ELISA is expandable to the standard array size (16×16 elements) normally used for production of arrays (e.g., by Genometrix Genomics, The Woodlands, Tex.), which would allow for the determination of 20 to 30 individual proteins within a single array. Polyclonal antibodies were used as detector antibodies in this array and no cross reactivity was detected, therefore, larger arrays made entirely of monoclonal antibodies should have no problem with cross-reactivity as well (possibly polyclonal detector antibodies will not encounter problems at greater densities either, so long as monoclonal capture antibodies are utilized exclusively).

Another advantage of this ELISA system is the fact that the loss of a single data point (probe value) does not negate the value of a test well. This is due to redundancy (capture antibodies printed in duplicate) and the use of several capture antibody concentrations. The use of regression equations formed from the titration of capture antibody has a balancing effect on occasional outlying data points without over or under emphasizing their impact on the total set as well.

The format of this assay is the standard 96 well glass slide array utilized for all arrays. This format is easily assimilated to automation, such as that for genotyping and gene expression.

A number of embodiments of the invention have been described. Nevertheless, it will be understood that various modifications may be made without departing from the spirit and scope of the invention. Accordingly, other embodiments are within the scope of the following claims.

What is claimed is:

1. A method for assembling protein arrays comprising the following steps:
(a) providing an array comprising a surface having a net positive or a net negative charge;
(b) providing a solution comprising a polypeptide having a net positive (cationic) charge density or a net positive charge polarity or a net negative (anionic) charge density or a net negative charge polarity at an amino terminal end or a carboxy terminal end; and
(c) adding the solution to the array under conditions allowing the positive or negative end of the polypeptide to align with the negative or positive charge of the array surface.

2. The method of claim 1, wherein the array surface comprises a net negative (anionic) charge density or a net negative charge polarity and the polypeptide comprises the net positive (cationic) charge density or the net positive charge polarity.

3. The method of claim 2, wherein hydroxyl groups on the array surface provide the net negative (anionic) charge density or the net negative charge polarity.

4. The method of claim 2, wherein sulfhydryl groups on the array surface provide the net negative (anionic) charge density or the net negative charge polarity.

5. The method of claim 1, wherein the array surface comprises a net positive (cationic) charge density or a net positive charge polarity and the polypeptide comprises a negative (anionic) charge density or a net negative charge polarity.

6. The method of claim 5, wherein the array surface comprises a net positive (cationic) charge density or a net positive charge polarity due to a plurality of charged amino groups on the array surface.

7. A method for assembling protein arrays comprising the following steps:
(a) providing an array;
(b) providing a solution comprising a polypeptide, wherein the solution comprises a viscosity sufficient to produce a surface tension such that the polypeptide-containing solution does not spread over an array surface area larger than about 200 nm in diameter; and
(c) adding the solution to the array.

8. A method for assembling protein arrays comprising the following steps:
(a) providing an array;
(b) providing a solution comprising a polypeptide, wherein the solution comprises a viscosity of between about 1 mN*s/m² to about 30 mN*s/m²; and
(c) adding the solution to the array.

9. The method of claim 7 or claim 8, wherein the solution comprises an organic polymer.

10. The method of claim 7 or claim 8, wherein the solution comprises glycerol.

11. The method of claim 7 or claim 8, wherein the solution comprises polyethylene glycol.

12. The method of claim 7 or claim 8, wherein the solution comprises sodium azide or sodium iodide.

13. The method of claim 12, wherein the sodium azide concentration is between about 0.2% and about 0.5%.

14. A method for assembling polypeptide arrays comprising the following steps:
(a) providing a substrate having a surface with a first net charge;
(b) providing a solution, wherein the solution includes a polypeptide having a net charge density or a net charge polarity for at least one of an amino terminal end or a carboxy terminal end; and,
(c) contacting the solution to the substrate.

15. The method of claim 14, wherein the first net charge comprises a negative (anionic) charge density or a negative charge polarity, and the net charge density of the polypeptide comprises a positive (cationic) charge density or the net charge polarity of the polypeptide comprises a positive charge polarity.

16. The method of claim 15, wherein at least one of hydroxyl groups or sulfhydryl groups on the substrate surface provide the first net charge with a negative (anionic) charge density or a negative charge polarity.

17. The method of claim 14, wherein the first net charge comprises a positive (cationic) charge density or a positive charge polarity, and the net charge density of the polypeptide comprises a negative (anionic) charge density or the net charge polarity of the polypeptide comprises a net negative charge polarity.

18. The method of claim 17, wherein amino groups on the substrate surface provide the first net charge with a positive (cationic) charge density or a positive charge polarity.

19. A method for assembling protein arrays comprising the following steps:
(a) providing a substrate;
(b) providing a solution comprising a polypeptide, wherein the solution has a viscosity sufficient to produce a surface tension such that the polypeptide-containing solution does not spread over a surface area larger than about 200 nm in diameter; and
(c) adding the solution to the substrate.

20. The method of claim 19, wherein the solution comprises an organic polymer.

21. The method of claim 19, wherein the solution comprises glycerol.

22. The method of claim 19, wherein the solution comprises polyethylene glycol.

23. The method of claim 19, wherein the solution comprises sodium azide or sodium iodide.

24. The method of claim 23, wherein the sodium azide concentration is between about 0.2% and about 0.5%.

25. A method for assembling protein arrays comprising the following steps:
(a) providing a substrate;
(b) providing a solution, wherein the solution includes a peptide and a viscosity of between about 1 mN*s/m² to about 30 mN*s/m²; and
(c) contacting the solution to the substrate.

26. The method of claim 25 wherein the solution comprises an organic polymer.

27. The method of claim 25, wherein the solution comprises glycerol.

28. The method of claim 25, wherein the solution comprises polyethylene glycol.

29. The method of claim 25, wherein the solution comprises sodium azide or sodium iodide.

30. The method of claim 29, wherein the sodium azide concentration is between about 0.2% and about 0.5%.

31. A method for assembling protein arrays comprising the following steps:
(a) providing a substrate having a surface with a net positive charge;
(b) providing a solution, wherein the solution includes a polypeptide having a net negative charge density or a net negative charge polarity for at least one of an amino terminal end or a carboxy terminal end; and,
(c) contacting the solution to the array.

32. The method of claim 31, wherein the solution comprises a viscosity sufficient to produce a surface tension such that the polypeptide-containing solution does not spread over an array surface area larger than about 200 nm in diameter.

33. The method of claim 31, wherein the solution comprises a viscosity of between about 1 mN*s/m² to about 30 mN*s/m².

34. The method of claim 31, wherein amino groups on the substrate surface produce the net positive charge by way of a net positive charge density or a net positive charge polarity.

35. A method for assembling protein arrays comprising the following steps:
(a) providing a substrate having a surface with a net negative charge;
(b) providing a solution, wherein the solution includes a polypeptide having a net positive (cationic) charge density or a net positive charge polarity for at least one of an amino terminal end or a carboxy terminal end; and,
(c) contacting the solution to the substrate.

36. The method of claim 35, wherein at least one of hydroxyl groups or sulfhydryl groups on the substrate surface produce the net negative charge by way of a net negative charge density or a net negative charge polarity.

37. The method of claim 36, wherein the solution comprises an organic polymer.

38. The method of claim 36, wherein the solution comprises glycerol.

39. The method of claim 36, wherein the solution comprises polyethylene glycol.

40. The method of claim 36, wherein the solution comprises sodium azide or sodium iodide.

41. The method of claim 40, wherein the sodium azide concentration is between about 0.2% and about 0.5%.

42. A method of claim 35, wherein the solution comprises a viscosity of between about 1 mN*s/m² to about 30 mN*s/m².

43. The method of claim 42, wherein the solution comprises an organic polymer.

44. The method of claim 42, wherein the solution comprises glycerol.

45. The method of claim 42, wherein the solution comprises polyethylene glycol.

46. The method of claim 42, wherein the solution comprises sodium azide or sodium iodide.

47. The method of claim 46, wherein the sodium azide concentration is between about 0.2% and about 0.5%.

48. The method of claim 35, wherein amino groups on the substrate surface provide a net positive charge density or a net positive charge polarity.

49. A method of making a polypeptide array on a substrate having a surface with a net charge, the method comprising contacting the surface with a liquid comprising a polypeptide selected or modified to have an opposite net charge at an end thereof.

50. A method of making an oriented polypeptide array, the method comprising
(a) selecting or modifying a plurality of polypeptides to have a net charge at an end thereof;
(b) selecting or modifying a substrate to have an opposite net charge on a surface thereof, and
(c) separately contacting aqueous liquids comprising one or more of the polypeptides with discrete regions of the surface.

51. A method of making an array comprising a substrate having a surface with a plurality of different polypeptides immobilized at discrete regions thereof, the method comprising contacting each region of the surface with a separate aqueous liquid comprising one of the polypeptides, wherein each polypeptide has an end having a net charge opposite the net charge of the surface.

52. A method for assembling polypeptide arrays, the method comprising:
(a) providing an array substrate having a surface with a net positive charge or a net negative charge;
(b) providing a solution comprising a polypeptide wherein at least one of an amino or carboxy terminal end of the polypeptide has one of i) a net positive (cationic) charge density, ii) a net positive charge polarity, iii) a net negative (anionic) charge density, or iv) a net negative charge polarity; and
(c) contacting the solution and the substrate under conditions allowing the end of the polypeptide to align with the charge of the surface.
